SEQUENCE LISTING (14 sequences)

SEQ ID NO: 1 - protein, 379 aa, Artificial Sequence
  synthetic construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 434 aa, Artificial Sequence
  synthetic construct
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 398 aa, Artificial Sequence
  synthetic construct
MGSTAILGLLLAVLQGVCAITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1143 bp, Artificial Sequence
  synthetic construct
attactcagcaagatctggcccctcagcaaagggccgctccacaacagaagagaagcagcccaagcgagggactctgccctcccggccaccacattagcgaggacggcagagattgcatcagctgtaagtacggccaagattacagcacacactggaatgatctgctgttctgtctgaggtgtacaaggtgcgactccggcgaagtcgaactgagcccttgcactactactaggaacactgtctgccagtgcgaggagggcacttttagggaggaggatagcccagagatgtgtaggaagtgtaggactggctgccctaggggcatggtgaaggtgggcgactgtactccatggagcgacattgagtgcgtgcataaggagagcggcactaagcacagcggcgaagtgccagccgtggaagagactgtgactagctcccccggcacaccagccagcgacaagacacacacttgtcctccttgcccagctccagaactgctcggaggaccaagcgtgtttctgttccctccaaagccaaaggacacactcatgatctctaggactccagaggtcacttgcgtggtcgtggatgtcagccacgaagatccagaggtgaagtttaattggtacgtggatggcgtggaggtccacaacgccaagacaaagcctagggaggagcagtacaatagcacttatagggtggtcagcgtgctgacagtgctgcaccaagattggctcaacggcaaggagtacaagtgcaaggtgagcaacaaggccctcccagcccctatcgagaagacaatctccaaggccaagggccaaccaagggagcctcaagtgtatacactccctcctagcagagacgagctcactaagaatcaagtgtctctgacttgtctggtgaagggcttttacccaagcgacatcgccgtggagtgggagagcaacggccaaccagagaacaactacaagacaactcctccagtgctggactccgacggcagcttcttcctctacagcaagctgactgtggataagtccagatggcagcaaggcaacgtcttcagctgtagcgtgatgcacgaggccctccacaaccactacacacagaagtctctgtctctgagccccggcaaataatag

SEQ ID NO: 5 - DNA, 1200 bp, Artificial Sequence
  synthetic construct
atggggtcaaccgccatccttggcctcctcctggctgttctccaaggagtctgtgccattactcagcaagatctggcccctcagcaaagggccgctccacaacagaagagaagcagcccaagcgagggactctgccctcccggccaccacattagcgaggacggcagagattgcatcagctgtaagtacggccaagattacagcacacactggaatgatctgctgttctgtctgaggtgtacaaggtgcgactccggcgaagtcgaactgagcccttgcactactactaggaacactgtctgccagtgcgaggagggcacttttagggaggaggatagcccagagatgtgtaggaagtgtaggactggctgccctaggggcatggtgaaggtgggcgactgtactccatggagcgacattgagtgcgtgcataaggagagcggcactaagcacagcggcgaagtgccagccgtggaagagactgtgactagctcccccggcacaccagccagcgacaagacacacacttgtcctccttgcccagctccagaactgctcggaggaccaagcgtgtttctgttccctccaaagccaaaggacacactcatgatctctaggactccagaggtcacttgcgtggtcgtggatgtcagccacgaagatccagaggtgaagtttaattggtacgtggatggcgtggaggtccacaacgccaagacaaagcctagggaggagcagtacaatagcacttatagggtggtcagcgtgctgacagtgctgcaccaagattggctcaacggcaaggagtacaagtgcaaggtgagcaacaaggccctcccagcccctatcgagaagacaatctccaaggccaagggccaaccaagggagcctcaagtgtatacactccctcctagcagagacgagctcactaagaatcaagtgtctctgacttgtctggtgaagggcttttacccaagcgacatcgccgtggagtgggagagcaacggccaaccagagaacaactacaagacaactcctccagtgctggactccgacggcagcttcttcctctacagcaagctgactgtggataagtccagatggcagcaaggcaacgtcttcagctgtagcgtgatgcacgaggccctccacaaccactacacacagaagtctctgtctctgagccccggcaaataatag

SEQ ID NO: 6 - protein, 440 aa, Artificial Sequence
  synthetic construct
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 7 - protein, 411 aa, Artificial Sequence
  synthetic construct
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 8 - protein, 435 aa, Artificial Sequence
  synthetic construct
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 399 aa, Artificial Sequence
  synthetic construct
MGSTAILGLLLAVLQGVCAITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1203 bp, Artificial Sequence
  synthetic construct
atgggctccaccgccatcctgggcctgctgctggccgtgctgcagggcgtgtgcgccattactcagcaagatctggcccctcagcaaagggccgctccacaacagaagagaagcagcccaagcgagggactctgccctcccggccaccacattagcgaggacggcagagattgcatcagctgtaagtacggccaagattacagcacacactggaatgatctgctgttctgtctgaggtgtacaaggtgcgactccggcgaagtcgaactgagcccttgcactactactaggaacactgtctgccagtgcgaggagggcacttttagggaggaggatagcccagagatgtgtaggaagtgtaggactggctgccctaggggcatggtgaaggtgggcgactgtactccatggagcgacattgagtgcgtgcataaggagagcggcactaagcacagcggcgaagtgccagccgtggaagagactgtgactagctcccccggcacaccagccagctccgacaagacacacacttgtcctccttgcccagctccagaactgctcggaggaccaagcgtgtttctgttccctccaaagccaaaggacacactcatgatctctaggactccagaggtcacttgcgtggtcgtggatgtcagccacgaagatccagaggtgaagtttaattggtacgtggatggcgtggaggtccacaacgccaagacaaagcctagggaggagcagtacaatagcacttatagggtggtcagcgtgctgacagtgctgcaccaagattggctcaacggcaaggagtacaagtgcaaggtgagcaacaaggccctcccagcccctatcgagaagacaatctccaaggccaagggccaaccaagggagcctcaagtgtatacactccctcctagcagagacgagctcactaagaatcaagtgtctctgacttgtctggtgaagggcttttacccaagcgacatcgccgtggagtgggagagcaacggccaaccagagaacaactacaagacaactcctccagtgctggactccgacggcagcttcttcctctacagcaagctgactgtggataagtccagatggcagcaaggcaacgtcttcagctgtagcgtgatgcacgaggccctccacaaccactacacacagaagtctctgtctctgagccccggcaaataatag

SEQ ID NO: 11 - protein, 373 aa, Artificial Sequence
  synthetic construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1125 bp, Artificial Sequence
  synthetic construct
attactcagcaagatctggcccctcagcaaagggccgctccacaacagaagagaagcagcccaagcgagggactctgccctcccggccaccacattagcgaggacggcagagattgcatcagctgtaagtacggccaagattacagcacacactggaatgatctgctgttctgtctgaggtgtacaaggtgcgactccggcgaagtcgaactgagcccttgcactactactaggaacactgtctgccagtgcgaggagggcacttttagggaggaggatagcccagagatgtgtaggaagtgtaggactggctgccctaggggcatggtgaaggtgggcgactgtactccatggagcgacattgagtgcgtgcataaggagagcggcactaagcacagcggcgaagtgccagccgtggaagagactgtgactagctcccccggcacacacacttgtcctccttgcccagctccagaactgctcggaggaccaagcgtgtttctgttccctccaaagccaaaggacacactcatgatctctaggactccagaggtcacttgcgtggtcgtggatgtcagccacgaagatccagaggtgaagtttaattggtacgtggatggcgtggaggtccacaacgccaagacaaagcctagggaggagcagtacaatagcacttatagggtggtcagcgtgctgacagtgctgcaccaagattggctcaacggcaaggagtacaagtgcaaggtgagcaacaaggccctcccagcccctatcgagaagacaatctccaaggccaagggccaaccaagggagcctcaagtgtatacactccctcctagcagagacgagctcactaagaatcaagtgtctctgacttgtctggtgaagggcttttacccaagcgacatcgccgtggagtgggagagcaacggccaaccagagaacaactacaagacaactcctccagtgctggactccgacggcagcttcttcctctacagcaagctgactgtggataagtccagatggcagcaaggcaacgtcttcagctgtagcgtgatgcacgaggccctccacaaccactacacacagaagtctctgtctctgagccccggcaaataatag

SEQ ID NO: 13 - protein, 23 aa, Artificial Sequence
  synthetic construct
VTSSPGTPASCDKTHTCPPCPAP

SEQ ID NO: 14 - protein, 7 aa, Artificial Sequence
  synthetic construct
ITQQDLA

(19) United States
(12) Patent Application Publication
(10) Pub. No.: US 2025/0011370 A1
(43) Pub. Date: Jan. 9, 2025

(54) THERAPEUTIC TREATMENT

(71) Applicants: UCL BUSINESS LTD, London (GB); UNIVERSITÄT ZU KÖLN, Köln (DE)

(72) Inventors: Richard BUTT, Cambridge (GB); Nadine CLEMO-EDWARDS, Cambridge (GB); Stephanie Jane HOPLEY, Cambridge (GB); Henning WALCZAK, London (GB)

(21) Appl. No.: 18/031,298

(22) PCT Filed: Oct. 8, 2021
(86) PCT No.: PCT/EP2021/077939

§ 371 Date: Apr. 11, 2023

(30) Foreign Application Priority Data
Oct. 9, 2020 (GB) 2016058.6

(51) Int. Cl. C07K 14/00; A61K 38/00; A61K 45/06; C12P 21/02

(52) U.S. Cl. CPC C07K 14/00; A61K 45/06; C12P 21/02; A61K 38/00

(57) ABSTRACT

The present invention relates to novel a novel fusion protein combining a portion of the TRAIL-R2 receptor with an Fc polypeptide region, and use of the same in methods of treatment of disease, for example in the treatment of cancer.

### CROSS-REFERENCE TO RELATED APPLICATIONS

This patent application is a national stage filing under 35 U.S.C. 371 of International Application No. PCT/EP2021/050577, filed Oct. 8, 2021, which claims the benefit of priority of GB 2016058.6 filed on 9 Oct. 2020 and both of which are herein incorporated in their entirety.

### SEQUENCE LISTING

This application is being filed electronically and includes an electronically submitted Sequence Listing in .txt format. The .txt file contains a sequence listing entitled “134541.00036_ST25 Sequence Listing.txt” created on Mar. 4, 2024 and is 36,203 bytes in size. The Sequence Listing contained in this.txt file is part of the specification and is hereby incorporated by reference herein in its entirety.

### TECHNICAL FIELD

The present invention relates generally to novel fusion proteins and their use in treating disease, such as cancer.

### BACKGROUND TO THE INVENTION

The Tumour Necrosis Factor (TNF) superfamily member TNF-related apoptosis-inducing ligand (TRAIL) can selectively induce apoptosis in a wide variety of tumour cells in vivo without causing toxicity to normal cells (Ashkenazi et al., 1999; Walczak et al., 1999). Consequently, these findings have triggered the design of TRAIL-receptor (TRAIL-R) agonistic cancer therapies (WO 2002094880 A1, U.S. Pat. No. 7,915,245 B2).

WO2004/085478 describes the design of fusion proteins based on TRAIL which comprise i) at least one first domain and a second domain which is heterologous to the first domain and ii) the selection of at least one terminal amino acid which is common to the first and the second domain, e.g. the last amino acid(s) of the first domain is (are) selected such that they are identical with the first amino acid(s) of the second domain. The disclosure describes an overlap which has a length of one, two or three amino acids. The intention is to provide a fusion protein which is free from a non-naturally occurring transition between the last amino acid of one domain and the first amino acid of another domain. One example is a TRAIL-R2-Fc polypeptide.

WO2015/001345 describes methods and materials for treating an individual with KRAS-mutated cancer or a cancer in which ROCK is inhibited independently of mutated KRAS. The invention described therein is based on the prevention or disruption of the binding of TRAIL-Receptor to its ligand, TRAIL, in vivo, for example by use of an agent that neutralises TRAIL and/or a the TRAIL-Receptor (such TRAIL-R2) and/or diminishes TRAIL/TRAIL-Receptor activity, thereby reducing cancer cell transformation, migration and metastasis, prolonging survival of patients. Preferred agents include the TRAIL-R2-Fc polypeptide described in WO2004/085478.

Other utilities for the TRAIL-R2-Fc polypeptide are described in WO2019/141862, where it is described for use in the treatment of inflammatory disease, in combination with other agents.

Although the TRAIL-R2-Fc polypeptide described in WO2004/085478 has many benefits as a pharmaceutical, nevertheless providing novel TRAIL-R2-Fc fusions with one or more advantages over those described in the prior art would provide a useful contribution to the art.

### DISCLOSURE OF THE INVENTION

The present invention relates to novel a novel TRAIL-R2-Fc polypeptide termed herein “TRAIL-R2-ΔC-Fc”. The primary sequence of TRAIL-R2-ΔC-Fc differs from the TRAIL-R2-Fc fusions of WO2004/085478 and WO2015001345. As demonstrated in the Examples hereinafter, this different sequence provides unexpected benefits in the context of its use as a pharmaceutical compared to other TRAIL-R2-Fc fusions.

One characteristic of the TRAIL-R2-ΔC-Fc of the invention compared to the prior art is that it has a modified overlap region in which the “C” (cysteine) is deleted (only sequence starting at Val 198 to Pro 220 is shown):

...VTSSPGTPAS[C]DKTHTCPPCPAP...

This actually creates a transition region which is less non-naturally occurring than those in the art discussed above, but nevertheless as described below has one or more benefits over previous TRAIL-R2-Fc fusions. In particular, modification of the TRAIL-R2-Fc sequence in this way has led to an unexpected improvement in expression of the protein, in combination with improved stability under pH, thermal and light stress conditions. The combination of these unexpected benefits enable improved manufacture over sequences described in the prior art whilst at the same time maintaining desired biological efficacy.

Thus the mature “TRAIL-R2-ΔC-Fc” polypeptide has the following sequence:

(Seq. ID No: 1)
ITQQDLAPQQRAAPQQKRSSPSEGLCPPGHHISEDGRDCISCKYGQDYST
HWNDLLFCLRCTRCDSGEVELSPCTTTRNTVCQCEEGTFREEDSPEMCRK
CRTGCPRGMVKVGDCTPWSDIECVHKESGTKHSGEVPAVEETVTSSPGTP
ASDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK..

The TRAIL-R2 portion is underlined. The Fc portion is depicted in bold. Note that there is a one amino acid overlap between the TRAIL-R2 portion and the human IgG1 Fc portion.

Because there are two splice forms of TRAIL-R2 expressed, and the splicing affects the extracellular domain of TRAIL-R2 (Screaton et al. 1997), at least two extracellular domains of TRAIL-R2 with differing amino acid sequences are known. In different embodiments, the TRAIL-binding portion of the extracellular domain of TRAIL-R2 can come from either one of these two when constructing TRAIL-inhibiting TRAIL-R2 fusion proteins. It will be understood that all aspects of the invention concerning TRAIL-R2-AC-Fc apply mutatis mutandis to these splice variant embodiments.

### Leader Sequences

The TRAIL-R2-ΔC-Fc may optionally comprise an N-terminal signal or leader sequence which allows secretion from a host cell after recombinant expression. The signal sequence may be a signal sequence which is homologous to the first domain of the fusion protein. Alternatively, the signal sequence may also be a heterologous signal sequence, e.g. the Igk or Igλ signal peptide sequence.

When produced recombinantly with a signal or leader sequence, the exact position of the N terminus can vary by a few amino acids; that means the mature protein described above can be, e.g. one to five amino acids shorter or longer at the N terminus.

Thus in one embodiment the TRAIL-R2-ΔC-Fc has a sequence including a leader sequence as follows. Here the leader peptide is depicted in italics. Again the mature protein starts with the sequence ITQQDLA.

(Seq. ID No: 2)
MEQRGQNAPAASGARKRHGPGPREARGARPGPRVPKTLVLVVAAVLLLVS
AESALITQQDLAPQQRAAPQQKRSSPSEGLCPPGHHISEDGRDCISCKYG
QDYSTHWNDLLFCLRCTRCDSGEVELSPCTTTRNTVCQCEEGTFREEDSP
EMCRKCRTGCPRGMVKVGDCTPWSDIECVHKESGTKHSGEVPAVEETVTS
SPGTPASDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPGK..

In another embodiment the TRAIL-R2-ΔC-Fc has the sequence as follows. Again a leader peptide is depicted in italics and the mature protein starts with the sequence ITQQDLA.

(Seq. ID No: 3)
MGSTAILGLLLAVLQGVCAITQQDLAPQQRAAPQQKRSSPSEGLCPPGHH
ISEDGRDCISCKYGQDYSTHWNDLLFCLRCTRCDSGEVELSPCTTTRNTV
CQCEEGTFREEDSPEMCRKCRTGCPRGMVKVGDCTPWSDIECVHKESGTK
HSGEVPAVEETVTSSPGTPASDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK..

Any of the TRAIL-R2-ΔC-Fc polypeptides or proteins discussed above may be referred to hereinafter as a “fusion protein of the invention”. Such fusions proteins may comprise, consist, or consist essentially of the mature or complete sequences described herein, or variants as described (e.g. based on splice variants of TRAIL-R2). Because the fusion proteins contain heterologous sequences, which do not occur together in nature, they are non-naturally occurring. Such fusion proteins (polypeptides) may be provided in isolated, purified, or semi-purified form.

As described below, the fusion proteins of the invention may be produced using any technique that provides for the production of recombinant and non-recombinant full length or functional fragments of these proteins by continuous cell lines in culture.

A further aspect of the present invention relates to a nucleic acid molecule encoding a fusion protein as described above. The nucleic acid molecule may be operatively linked to an expression control sequence, e.g. an expression control sequence which allows expression of the nucleic acid molecule in a desired host cell. The nucleic acid molecule may be located on a vector, e.g. a plasmid, a bacteriophage, a viral vector, a chromosomal integration vector, etc.

The resulting proteins may be used with or without modifications such as labelling, recombinant joining to further polypeptides or with molecules functioning as reporters. Modifications can be covalent and/or non-covalent.

Further, the invention relates to a cell or non-human organism transformed or transfected with a nucleic acid molecule as described above. Such transgenic organisms may be generated by known methods of genetic transfer including homologous recombination.

A further aspect of the present invention relates to a pharmaceutical composition comprising as an active agent at least one fusion protein as described above.

Also described are uses of, or methods employing, the fusions of the invention in therapy e.g. for treating cancer or other diseases.

Some of these aspects and embodiments of the invention will now be described in more detail:

### Utility in Therapy

In one aspect the invention provides a method of therapeutic treatment of an individual (e.g. of a disease or disorder, the terms are used interchangeable) the method comprising administering to the individual a therapeutically-effective amount of a fusion protein of the invention. Also provided is use of a fusion protein of the invention for treatment by therapy of the human or animal body, or use of a fusion protein of the invention for preparation of a medicament for this purpose. Discussion of methods of treatment hereinafter will be understood to apply mutatis mutandis to these uses, and vice versa.

### Individuals or Subjects

The individual to be treated will typically be a mammal e.g. a human or non-human mammal.

Thus in one embodiment the individual is a human subject e.g. a patient.

The mammal may be a non-human mammal e.g. a test animal such as a rodent (e.g. mouse, rat) or primate. The mammal may be a transgenic mammal.

The subject or organism may be a bird, fish, reptile or amphibian.

The specification herein defines methods of treating diseases as discussed herein using a fusion protein of the invention.

Further disclosed herein is a fusion protein of the invention for use in the treatment of those diseases.

Likewise, further disclosed herein are use of the corresponding agents or combinations of agents in the preparation of a medicament for use in the treatment of those diseases.

### Examples of Target Diseases

In one embodiment the cancer is a KRAS-mutated cancer.

Oncogenic mutation of KRAS is very frequent in pancreatic cancer (Hidalgo, 2010; Jaffee et al., 2002), frequent in colon (Grady and Markowitz, 2002) and lung cancer (Mitsuuchi and Testa, 2002) and also occurs, albeit at much lower frequencies, in other cancer types such as biliary tract malignancies, endometrial cancer (Ito et al., 1996), cervical cancer (Wegman et al., 2011), bladder cancer (Przybojewska et al., 2000), liver cancer and cholangiocarcinoma (Boix-Ferrero et al., 2000), myeloid leukemia (Ahmad et al., 2009) and breast cancer (Karnoub and Weinberg, 2008). These are some of the most aggressive human cancers and despite many efforts to design efficient therapies for them, survival rates of patients with cancers that bear oncogenic KRAS mutations are still very low. In some cases oncogenic mutation of KRAS is even an exclusion criterion for treatment by certain drugs because they have been found to be ineffective in these patients (Amado et al., 2008; Deschoolmeester et al., 2010; Karapetis et al., 2008; van Krieken et al., 2008). The aggressive behaviour of KRAS-mutated cancers can be attributed to their inherent chemo-resistance, strong invasiveness and capacity to metastasize (Downward, 2003), traits which render these cancers very difficult to treat (Chaffer and Weinberg, 2011).

Thus the fusion proteins of the invention may be used to reduce cancer cell transformation, migration and metastasis in KRAS-mutated cancer patients.

By way of non-limiting example, the present invention may be applied to KRAS-mutated patients with pancreatic cancer, colon cancer, lung cancer, breast cancer, endometrial cancer, cervical cancer, liver cancer, myeloid leukemia, cholangiocarcinoma or bladder cancer. Other preferred cancers are described hereinafter. The most preferred target cancers are KRAS-mutated pancreatic, colon or lung cancers.

WO2015/001345 teaches that oncogenic alterations that result in inhibition of ROCK can also enable TRAIL/TRAIL-R-mediated Rac1 activation. Besides mutated KRAS, integrin signalling through Src and FAK (Ahn et al., 2010), BRAF (Klein et al., 2008), Raf-1 (Ehrenreiter et al., 2009) and Notch3 (Belin de Chantemele et al., 2008) have been linked to ROCK inhibition.

Therefore, cancers that present with aberrant signalling in the above named pathways may also employ the TRAIL/TRAIL-R2 system for enhanced migration and metastasis, and may be treated with the fusion proteins of the present invention.

Other cancers in which the fusion proteins of the present invention may be used include non-KRAS associated cancers. These include, but are not limited to CRC, squamous cell carcinoma, breast cancer and NSCLC (Bozkurt et al., J Cell Biol 2021; Ethiraj et al., J Cell Physiol 2020; Yan et al., J Cancer Res Clin Oncol 2012).

Other diseases in which the fusion proteins of the present invention may be used include:

Pulmonary arterial hypertension (Hameed et al., J Exp Med 2012; Lawrie, Vascul Pharmacol 2014);

Non-alcoholic steatohepatitis and other liver disease (Hirsova et al., Hepatol Commun 2017; Farrell et al., J Gastroenterol Hepatol 2009; Johnson Valiente et al., J Infect Dis 2021);

Neurodegeneration and neuroinflammation (Qin et al., Int J Mol Sci 2021);

Inflammatory disease including arthritis (Jeong et al., ELife 2021), inflammatory bowel disease, Crohn's disease and ulcerative colitis (Wu et al., Sci Rep 2019),

Chronic lung disease (Starkey et al., Mucosal Immunol 2014)

### Dosage and Administration

The fusion protein of the invention will generally be administered as a pharmaceutical composition comprising an effective amount to achieve the intended purpose, when suitably administered to an individual.

The determination of an effective dose is well within the capability trained personnel. For any compounds, the therapeutically effective dose can be estimated initially either in cell culture assays, e.g., of cell lines or in animal models, usually but not exclusively mice. The animal model may also be used to determine the appropriate concentration range and route of administration. Based on such pilot experiments, useful doses and routes for administration in humans can be determined. A therapeutically effective dose refers to that amount of active ingredient, for example fusion protein of the invention, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as LD50/ED50. Pharmaceutical compositions, which exhibit large therapeutic indices, are preferred. The dosage is preferably within a range of circulating concentrations that include the ED50 with little or no toxicity. The dosage varies within this range depending upon the dosage employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy.

Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells and conditions as detailed above.

Generally, a daily dose of between 0.01 μg/kg of body weight and 100 mg/kg of body weight of agent according to the invention may be used for treating, ameliorating, or preventing disease. More preferably, the daily dose of agent is between 1 mg/kg of body weight and 100 mg/kg of body weight, more preferably between 10 mg/kg and 10 mg/kg body weight, and most preferably between approximately 100 mg/kg and 10 mg/kg body weight.

The duration of treatment may be:
1 to 14, e.g. 1, 2, 3, 4, 5, 6, 7, 8, 9, 10, 11, 12, 13 or 14 days, or
1 to 4, e.g. 1, 2, 3 or 4 weeks, or
1 to 12, e.g. 1, 2, 3, 4, 5, 6, 7, 9, 9, 10, 11, or 12 months.

For prophylaxis, the treatment may be ongoing.

In some embodiments the agent or agents may be administered to a subject or individual during late-stage disease.

In all cases the treatment duration will generally be subject to advice and review of the physician.

In one embodiment, the dosage of the fusion protein may be 20-500 mg twice per week, weekly, every ten days, bi-weekly, every three weeks, or every four weeks.

Most preferably the given dose will be between 50 mg and 200 mg twice per week, weekly, or bi-weekly.

The precise nature of the carrier or other material combined with the fusion protein of the invention for pharmaceutical use will depend on the route of administration, which may be by bolus, infusion, injection or any other suitable route, as discussed below.

The pharmaceutical compositions detailed in this invention may be administered by any number of routes including, but not limited to, intravenous, intramuscular, intra-arterial, intramedullary, intrathecal, intraventricular, transdermal, subcutaneous, intraperitoneal, intranasal, enteral, topical, sublingual or rectal means.

A preferred mode of administration will be parenteral, for example sub-cutaneous or intravenous administration.

For parenteral administration, e.g. by injection, the pharmaceutical composition comprising the fusion protein of the invention may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilisers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

### Use of the Fusions for Inhibition of TRAIL

In another aspect, the invention provides a method or a use of a fusion protein of the invention to bind TRAIL or as a TRAIL-inhibiting agent, for example to neutralise TRAIL and/or a TRAIL receptor which is optionally TRAIL-R1 and/or TRAIL-R2 and/or diminish the interaction of TRAIL with TRAIL-R1 and/or TRAIL-R2 and/or the activity resulting from these interactions.

Such use or method may be in vitro or may form part of treatment of an individual by administering the fusion protein of the invention to the individual.

“Neutralises” in this context will be understood to mean modulates a biological activity of, either directly (for example by binding to the relevant target) or indirectly. As used herein, the term “biological activity” means any observable effect resulting from the interaction between the protein\receptor (binding partners). Non-limiting examples of biological activity in the context of the present invention include signalling and regulation of the genes discussed herein, e.g. those involved in cell death by apoptosis, necroptosis and/or pyroptosis or in activation of genes that lead to the expression of cytokines, including chemokines.

“Neutralises” does not imply complete inactivation. The modulation is generally inhibition i.e. a reduction or diminution in the relevant biological activity by comparison with the activity seen in the absence of the agent.

Neutralisation is typically achieved by (i) preventing or inhibiting the ligand from binding to the receptor; (ii) disrupting the receptor/ligand complex resulting from such binding; (iii) preventing or inhibiting the activity of factors and/or enzymes responsible for mediating the biological activity normally induced by the ligand-induced stimulation of the receptor(s).

### Pharmaceutical Compositions

The agents utilised in the present invention may be provided as a “pharmaceutical composition” (e.g., formulation, preparation, medicament) comprising one or more agents described herein, and a pharmaceutically acceptable carrier, diluent, or excipient.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition comprising at least one fusion protein of the invention, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

### Combination Therapies

In some embodiments the methods or treatments of the present invention may be combined with other therapies, whether symptomatic or disease modifying e.g. a second therapeutic agent believed to show therapeutic benefit in the relevant cancers.

The term “treatment” includes combination treatments and therapies, in which two or more treatments or therapies are combined, for example, sequentially or simultaneously.

For example it may be beneficial to combine treatment with a fusion protein of the invention as described herein with one or more other (e.g., 1, 2, 3, 4) agents or therapies.

Appropriate examples of co-therapeutics will be known to those skilled in the art on the basis of the disclosure herein. Typically the co-therapeutic may be any known in the art which it is believed may give therapeutic effect in treating the cancers described herein e.g. KRAS oncogenic related cancers.

Thus in one embodiment of this invention, the fusion protein of the invention is applied to an individual with a cancer (e.g. pancreatic cancer, colon cancer, lung cancer, breast cancer, endometrial cancer, cervical cancer, liver cancer, myeloid leukemia, cholangiocarcinoma or bladder cancer) in combination with a standard chemo-/and/or radiotherapy that is given in the respective cancer [e.g. cisplatin, Carboplatin, etoposide, gemcitabine, Vinorelbine,, Paclitaxel (Taxol), Docetaxel (Taxotere), Doxorubicin, Pemetrexed, Fluorouracil (also called 5FU), Capecitabine, Oxaliplatin, Irinotecan (Camptothecin), Uftoral (also called tegafur with uracil), folinic acid, Cyclophosphamide, Epirubicin. Methotrexate, Mitomycin, Mitozantrone, or any combination thereof]

In one embodiment the fusion protein of the invention is applied to an individual with a cancer which is KRAS-mutated, and the standard chemo-/and/or radiotherapy is e.g. cisplatin, Carboplatin, etoposide, gemcitabine, Vinorelbine,, Paclitaxel (Taxol), Docetaxel (Taxotere), Doxorubicin, Pemetrexed, Fluorouracil (also called 5FU), Capecitabine, Oxaliplatin, Irinotecan (Camptothecin), Uftoral (also called tegafur with uracil), folinic acid, Cyclophosphamide, Epirubicin. Methotrexate, Mitomycin, Mitozantrone, or any combination thereof.

Where the cancer is KRAS-mutated pancreatic, colon and lung cancers the standard chemo-/and/or radiotherapy is e.g. cisplatin, Carboplatin, etoposide, gemcitabine, Vinorelbine,, Paclitaxel (Taxol), Docetaxel (Taxotere), Doxorubicin, Pemetrexed, Fluorouracil (also called 5FU), Capecitabine, Oxaliplatin, Irinotecan (Camptothecin), Uftoral (also called tegafur with uracil), folinic acid, or any combination thereof.

The particular combination would be at the discretion of the physician who would also select dosages using his/her common general knowledge and dosing regimens known to a skilled practitioner.

The agents (i.e., fusion protein of the invention, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., fusion protein of the invention, plus one or more other agents) may be formulated together in a single dosage form, or alternatively, the individual agents may be formulated separately and presented together in the form of a kit, optionally with instructions for their use.

### Detection of KRAS Mutations and Kits

The invention may comprise screening patients for the KRAS or other mutation (e.g. by PCR of a sample from the individual) in order to select or reject them for treatment with the agents described herein (“companion diagnostics”).

A commercially available diagnostic kit for detecting mutations in the KRAS oncogene is, for example, the TheraScreen™ K-Ras mutation detection kit, for detecting the mutations 12Ala, 12Asp, 12Arg, 12Cys, 12Ser, 12Val and 13Asp.

A diagnostic kit for detecting mutations in the KRAS oncogene is, for example, the TheraScreen™ KRAS PCR kit by Qiagen.

Another, and preferred commercially available diagnostic kit herein for identifying mutations in the KRAS gene is the cobas™ KRAS Mutation Test by Roche (http://molecular.roche.com/assays/Pages/cobasKRASMutationTest.aspx), which is a real-time PCR test that can be used for detecting a broad spectrum of mutations in codons 12, 13 and 61 of the KRAS gene, covering the mutations 12D, 12V, 12C, 12A, 12S, 12R, 12F, 13D, 13C, 13R, 13S, 13A, 13V, 131, 61H, 61L, 61R, 61K, 61E and 61P.

This kit was observed to yield best results in formalin fixed tissues (Gonzalez de Castro et al., 2012).

For mutational testing a typical cancer (tumour) sample comprising nucleic acid is used, which may be selected from the group consisting of a tissue, a biopsy probe, cell lysate, cell culture, cell line, organ, organelle, biological fluid, blood sample, urine sample, skin sample, and the like.

The present invention further provides the use of such KRAS mutation kits as companion diagnostic to the methods of the invention i.e. treatments utilising a fusion protein of the invention.

The present invention further includes the use of such kits for determining likelihood of effectiveness of treatment by the fusion proteins of the invention, optionally in combination with one or more other anti-cancer agents, in a mammalian, preferably human, patient diagnosed with cancer (such as KRAS-mutated cancers described herein), said kit preferably comprising means for detecting one or more mutations in the KRAS oncogene described herein.

### Nucleic Acids, Vectors and Cells

A further aspect of the present invention relates to a nucleic acid molecule encoding a fusion protein of then invention. The nucleic acid molecule may be a DNA molecule, e.g. a double-stranded or single-stranded DNA molecule, or an RNA molecule.

The nucleic acid molecule may encode the mature fusion protein or a precursor thereof, e.g. a pro-or pre-proform of the fusion protein which may comprise a signal or leader sequence or other heterologous amino acid portions for secretion or purification which are preferably located at the N-and/or C-terminus of the fusion protein. The heterologous amino acid portions may be linked to the first and/or second domain via a protease cleavage site, e.g. a Factor Xₐ, thrombin or IgA protease cleavage site. An example TRAIL-R2-ΔC-Fc nucleotide sequence encoding the mature protein of Seq. ID No: 1 is as follows:

(Seq. ID No: 4)
ATTACTCAGCAAGATCTGGCCCCTCAGCAAAGGGCCGCTCCACAACAGAA
GAGAAGCAGCCCAAGCGAGGGACTCTGCCCTCCCGGCCACCACATTAGCG
AGGACGGCAGAGATTGCATCAGCTGTAAGTACGGCCAAGATTACAGCACA
CACTGGAATGATCTGCTGTTCTGTCTGAGGTGTACAAGGTGCGACTCCGG
CGAAGTCGAACTGAGCCCTTGCACTACTACTAGGAACACTGTCTGCCAGT
GCGAGGAGGGCACTTTTAGGGAGGAGGATAGCCCAGAGATGTGTAGGAAG
TGTAGGACTGGCTGCCCTAGGGGCATGGTGAAGGTGGGCGACTGTACTCC
ATGGAGCGACATTGAGTGCGTGCATAAGGAGAGCGGCACTAAGCACAGCG
GCGAAGTGCCAGCCGTGGAAGAGACTGTGACTAGCTCCCCCGGCACACCA
GCCAGCGACAAGACACACACTTGTCCTCCTTGCCCAGCTCCAGAACTGCT
CGGAGGACCAAGCGTGTTTCTGTTCCCTCCAAAGCCAAAGGACACACTCA
TGATCTCTAGGACTCCAGAGGTCACTTGCGTGGTCGTGGATGTCAGCCAC
GAAGATCCAGAGGTGAAGTTTAATTGGTACGTGGATGGCGTGGAGGTCCA
CAACGCCAAGACAAAGCCTAGGGAGGAGCAGTACAATAGCACTTATAGGG
TGGTCAGCGTGCTGACAGTGCTGCACCAAGATTGGCTCAACGGCAAGGAG
TACAAGTGCAAGGTGAGCAACAAGGCCCTCCCAGCCCCTATCGAGAAGAC
AATCTCCAAGGCCAAGGGCCAACCAAGGGAGCCTCAAGTGTATACACTCC
CTCCTAGCAGAGACGAGCTCACTAAGAATCAAGTGTCTCTGACTTGTCTG
GTGAAGGGCTTTTACCCAAGCGACATCGCCGTGGAGTGGGAGAGCAACGG
CCAACCAGAGAACAACTACAAGACAACTCCTCCAGTGCTGGACTCCGACG
GCAGCTTCTTCCTCTACAGCAAGCTGACTGTGGATAAGTCCAGATGGCAG
CAAGGCAACGTCTTCAGCTGTAGCGTGATGCACGAGGCCCTCCACAACCA
CTACACACAGAAGTCTCTGTCTCTGAGCCCCGGCAAATAATAG..

An example TRAIL-R2-ΔC-Fc nucleotide sequence encoding a the fusion including a leader peptide as shown in Seq ID No: 3 is as follows:

(Seq. ID No: 5)
ATGGGGTCAACCGCCATCCTTGGCCTCCTCCTGGCTGTTCTCCAAGGAGT
CTGTGCCATTACTCAGCAAGATCTGGCCCCTCAGCAAAGGGCCGCTCCAC
AACAGAAGAGAAGCAGCCCAAGCGAGGGACTCTGCCCTCCCGGCCACCAC
ATTAGCGAGGACGGCAGAGATTGCATCAGCTGTAAGTACGGCCAAGATTA
CAGCACACACTGGAATGATCTGCTGTTCTGTCTGAGGTGTACAAGGTGCG
ACTCCGGCGAAGTCGAACTGAGCCCTTGCACTACTACTAGGAACACTGTC
TGCCAGTGCGAGGAGGGCACTTTTAGGGAGGAGGATAGCCCAGAGATGTG
TAGGAAGTGTAGGACTGGCTGCCCTAGGGGCATGGTGAAGGTGGGCGACT
GTACTCCATGGAGCGACATTGAGTGCGTGCATAAGGAGAGCGGCACTAAG
CACAGCGGCGAAGTGCCAGCCGTGGAAGAGACTGTGACTAGCTCCCCCGG
CACACCAGCCAGCGACAAGACACACACTTGTCCTCCTTGCCCAGCTCCAG
AACTGCTCGGAGGACCAAGCGTGTTTCTGTTCCCTCCAAAGCCAAAGGAC
ACACTCATGATCTCTAGGACTCCAGAGGTCACTTGCGTGGTCGTGGATGT
CAGCCACGAAGATCCAGAGGTGAAGTTTAATTGGTACGTGGATGGCGTGG
AGGTCCACAACGCCAAGACAAAGCCTAGGGAGGAGCAGTACAATAGCACT
TATAGGGTGGTCAGCGTGCTGACAGTGCTGCACCAAGATTGGCTCAACGG
CAAGGAGTACAAGTGCAAGGTGAGCAACAAGGCCCTCCCAGCCCCTATCG
AGAAGACAATCTCCAAGGCCAAGGGCCAACCAAGGGAGCCTCAAGTGTAT
ACACTCCCTCCTAGCAGAGACGAGCTCACTAAGAATCAAGTGTCTCTGAC
TTGTCTGGTGAAGGGCTTTTACCCAAGCGACATCGCCGTGGAGTGGGAGA
GCAACGGCCAACCAGAGAACAACTACAAGACAACTCCTCCAGTGCTGGAC
TCCGACGGCAGCTTCTTCCTCTACAGCAAGCTGACTGTGGATAAGTCCAG
ATGGCAGCAAGGCAACGTCTTCAGCTGTAGCGTGATGCACGAGGCCCTCC
ACAACCACTACACACAGAAGTCTCTGTCTCTGAGCCCCGGCAAATAATA
G..

The nucleic acid molecule may be operatively linked to an expression control sequence, e.g. an expression control sequence which allows expression of the nucleic acid molecule in a desired host cell. The nucleic acid molecule may be located on a vector, e.g. a plasmid, a bacteriophage, a viral vector, a chromosomal integration vector, etc.

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. Molecular biology techniques suitable for the expression of polypeptides in cells are well known in the art. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

An expression vector as used herein is a DNA molecule used to transfer and express foreign genetic material in a cell. Such vectors include a promoter sequence operably linked to the gene encoding the protein to be expressed. “Promoter” means a minimal DNA sequence sufficient to direct transcription of a DNA sequence to which it is operably linked. “Promoter” is also meant to encompass those promoter elements sufficient for promoter-dependent gene expression controllable for cell type specific expression; such elements may be located in the 5′ or 3′ regions of the native gene. The term “operably linked” used herein includes the situation where a selected sequence to be expressed and promoter are covalently linked in such a way as to place the expression of the gene under the influence or control of the promoter.

An expression vector may also include a termination codon and expression enhancers.

Any suitable vectors, promoters, enhancers and termination codons may be used to express the fusion protein of the invention from an expression vector according to the invention. Suitable vectors include plasmids, binary vectors, phages, phagemids, viral vectors and artificial chromosomes (e.g. yeast artificial chromosomes or bacterial artificial chromosomes). As described in more detail below, preferred expression vectors include viral vectors such as lentiviral vectors.

An expression vector may additionally include a reporter gene encoding a reporter protein. An example of a reporter protein is a green fluorescent protein (GFP). A reporter gene may be operably linked to its own promoter or, more preferably, may be operably linked to the same promoter as the polynucleotide encoding the fusion protein of the invention. As an example of the latter, polynucleotide encoding the fusion protein of the invention and reporter gene may be located either side of a sequence encoding a 2A peptide, such as a T2A peptide. 2A peptides are short (˜20 amino acids) sequences that permit multicistronic gene expression from single promoters by impairing peptide bond formation during ribosome-mediated translation.

Various expression vector/host cell systems may be used to express the nucleic acid sequences encoding the fusion proteins of the present invention. Suitable host cells include, but are not limited to, prokaryotic cells such as bacteria, e.g. E.coli, eukaryotic host cells such as yeast cells, insect cells, plant cells or animal cells, and mammalian (e.g. human cells).

The resulting proteins may be used with or without modifications such as labelling, recombinant joining to further polypeptides or with molecules functioning as reporters. Modifications can be covalent and/or non-covalent.

Further, the invention relates to a cell non-human organism transformed or transfected with a nucleic acid molecule as described above. Such transgenic organisms may be generated by known methods of genetic transfer including homologous recombination.

Wherever amino acid and nucleic acid sequences are discussed herein (for example in respect of coding fusion proteins), it will be appreciated by the skilled technician that functional derivatives of the amino acid, and nucleic acid sequences, disclosed herein, are also envisaged-such derivatives may have a sequence which has at least 70%, more preferably 75%, even more preferably 85%, and even more preferably 90% to any of the sequences referred to is also envisaged. Preferably, the amino acid/polypeptide/nucleic acid sequence has 92% identity, even more preferably 95% identity, even more preferably 97% identity, even more preferably 98% identity and, most preferably, 99% identity with any of the referred to sequences. In each case the fusion protein has a modified overlap region in which the “C” (a free cysteine) is deleted as described above.

Calculation of percentage identities between different amino acid/polypeptide/nucleic acid sequences may be carried out as follows. A multiple alignment is first generated by the ClustalX program (pair wise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Alternatively, a substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2× SSC/0.1% SDS at approximately 5-65° C. Alternatively, a substantially similar polypeptide may differ by at least 1, but less than 5, 10, 20, 50 or 100 amino acids from the peptide sequences according to the present invention.

Due to the degeneracy of the genetic code, it is clear that any nucleic acid sequence could be varied or changed without substantially affecting the sequence of the protein encoded thereby, to provide a functional variant thereof. Degeneratively equivalent (e.g. codon optimised) nucleotide sequences to any of those described herein may of course be used in their place.

Suitable nucleotide variants are those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

The accurate alignment of protein or DNA has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Frequently, automatically generated alignments require manual alignment, exploiting the trained user's knowledge of the protein family being studied, e.g., biological knowledge of key conserved sites. One such alignment editor programs is Align (http://www.gwdg.de/˜dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYLIP.

Other modifications in protein sequences are also envisaged and within the scope of the claimed invention, i.e. those which occur during or after translation, e.g. by acetylation, amidation, carboxylation, phosphorylation, proteolytic cleavage or linkage to a ligand.

### Modification of Polypeptides

It will be appreciated that fusion proteins used or provided according to the invention may be derivatives of native or original sequences, and thus include derivatives that increase the effectiveness or half-life of the agent in vivo. Examples of derivatives capable of increasing the half-life of polypeptides according to the invention include peptoid derivatives, D-amino acid derivatives and peptide-peptoid hybrids.

Fusion proteins according to the present invention may be subject to degradation by a number of means (such as protease activity at a target site). Such degradation may limit their bioavailability and hence therapeutic utility. There are a number of well-established techniques by which peptide derivatives that have enhanced stability in biological contexts can be designed and produced. Such peptide derivatives may have improved bioavailability as a result of increased resistance to protease-mediated degradation. Preferably, a derivative suitable for use according to the invention is more protease-resistant than the protein or peptide from which it is derived. Protease-resistance of a peptide derivative and the protein or peptide from which it is derived may be evaluated by means of well-known protein degradation assays. The relative values of protease resistance for the peptide derivative and peptide may then be compared.

Peptoid derivatives of fusion proteins according to the invention may be readily designed from knowledge of the structure of the TRAIL receptor or Fc region. Commercially available software may be used to develop peptoid derivatives according to well-established protocols.

Retropeptoids, (in which all amino acids are replaced by peptoid residues in reversed order) are also able to mimic proteins or peptides according to the invention. A retropeptoid is expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

A further embodiment of a modified form of peptides or proteins according to the invention comprises D-amino acid forms. In this case, the order of the amino acid residues is reversed. The preparation of peptides using D-amino acids rather than L-amino acids greatly decreases any unwanted breakdown of such derivative by normal metabolic processes, decreasing the amounts of the derivative which needs to be administered, along with the frequency of its administration.

### General Statements

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress (prolonged survival), a halt in the rate of progress, regression of the condition, amelioration of the condition, and cure of the condition. The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen.

The invention also embraces treatment as a prophylactic measure is also included and “treating” will be understood accordingly. Prophylactic treatment may utilise a “prophylactically effective amount,” which, where used herein, pertains to that amount of an agent which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

Wherever a method of treatment employing an agent is described herein, it will be appreciated that an agent (any one of the first, second, third agents) for use in that method is also described, as is an agent (any one of the first, second, third agents) for use in the manufacture of a medicament for treating the relevant inflammatory disease. Also described is any one of the first, second, third agents for use in methods of enhancing the activity of the other two agents.

Wherever a composition is described herein, it will be appreciated that the same composition for use in the therapeutic methods (including prophylactic methods) described herein is also envisaged, as is the composition for use in the manufacture of a medicament for treating the relevant inflammatory disease.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

### FIGURE

FIG. 1: Altering the linker does not affect the biological activity of the Fc fusion protein, as discussed in Example 5.

### SEQUENCES

Other sequences referred to herein are shown in the Sequence Annex.

### EXAMPLES

### Example 1: Expression and Purification of the Recombinant TRAIL-R2-Fc Fusion Proteins

cDNAs corresponding to the appropriate DNA sequence were cloned into standard vector systems using conventional (non-PCR) cloning techniques. Vector plasmids were gene synthesised. Plasmid DNA was prepared under low-endotoxin conditions based on anion exchange chromatography. DNA concentration was determined by measuring the absorption at a wavelength of 280 nm. Correct sequences were verified by Sanger sequencing.

Expression plasmids encoding the TRAIL-R2-Fc fusion protein were transfected into CHO-K1 cells and expressed in appropriately size shake flask cultures to produce Fc fusion protein.

Supernatant was harvested using centrifugation and subsequent filtration (0.2 μM filter). The Fc fusion protein was purified using protein A based purification.

The concentration was determined by measuring absorption at a wavelength of 280 nm. Purity was determined by analytical size exclusion chromatography using an Agilent AdvanceBio SEC column and using DPBS as a running buffer. Endotoxin content was measured using standard methods by using a Charles River Endosafe PTS system.

Example expression levels are shown in Table 1.

TABLE 1
Comparison of the expression of the recombinant
TRAIL-R2-Fc fusion proteins

| | | |
|---|---|---|
| Molecule | Expression | SEC (% monomer) |

| | | | |
|---|---|---|---|
| TRAIL-R2-Fc | 150 | mg/L | 99% |
| TRAIL-R2-Fc ΔC153 | 210 | mg/L | 99% |
| TRAIL-R2-Fc C153S | 65 | mg/L | <95% |
| TRAIL-R2-Fc ΔTPASCDK | 130 | mg/L | 98% |

Thus improved expression levels were observed with the TRAIL-R2-FcΔC Fc fusion protein compared with all other variants.

### Example 2: TRAIL-R2-FcΔC Molecule Shows Better Stability under Thermal Stress in Neutral pH Buffer

Material was thawed and both a concentration measurement and analytical size exclusion chromatography (aSEC) were carried out. The material was then exposed to thermal stress by heating to 40 C for 2 weeks. Samples were evaluated following incubation under stressed and unstressed conditions.

Molecules were subjected to analytical size exclusion chromatography (aSEC) to quantify the levels of (1) the percentage of high molecular weight species, (% HMwS) that is indicative of aggregation risk; (2) the percentage of low molecular weight species, (% HMwS) that is indicative of fragmentation risk; (3) the percentage monomer that is indicative of monomeric non-degraded species.

For the aSEC assay: Samples at 4-5 mg/ml were injected at 10 μl onto two TSKgel super SW3000 4.6×300 mm, 4 μm columns connected in series, equilibrated in a sodium phosphate running buffer containing NaCl. The flow rate was 0.2 ml/min and UV detection was at 214 nm and 280 nm. Chromatograms were automatically processed and integrated. Manual re-integration was carried out where necessary to account for trailing peaks.

Table 2 shows a comparison between the Fc fusion proteins which had been subjected to thermal stress.

TABLE 2
TRAIL-R2-FcΔC molecule shows better stability
under thermal stress in neutral pH buffer

| | % total | % Total | % total |
|---|---|---|---|
| Sample | HMwS | monomer | LMwS |
| TRAIL-R2-Fc | 0.57 | 97.70 | 1.73 |
| TRAIL-R2-Fc ΔC153 | 0.55 | 97.74 | 1.71 |
| TRAIL-R2-Fc ΔTPASCDK | 0.54 | 98.08 | 1.38 |

Thus improved thermal stability could be observed with the TRAIL-R2-FcΔC Fc fusion protein compared with the TRAIL-R2-Fc variant.

Further studies were conducted to compare TRAIL-R2-FcΔC to TRAIL-R2-Fc C153S.

Table 3 shows a comparison between TRAIL-R2-FcΔC and TRAIL-R2-Fc C153S when placed under thermal stress in neutral buffer. After incubation in neutral buffer at 40C for 2W, 0.9% main peak decreases were observed in TRAIL-R2-FcΔC, and 1.0% main peak decreases were observed in TRAIL-R2-Fc C153S.

TABLE 3
Comparison of TRAIL-R2-FcΔC and TRAIL-R2-Fc C153S
molecules under thermal stress in neutral pH buffer

| | TRAIL-R2-FcΔC | | | TRAIL-R2-Fc C153S | | |
|---|---|---|---|---|---|---|
| | % HMwS | % monomer | % LMwS | % HMwS | % monomer | % LMwS |
| 40 C. 1 | 1.2 | 98.5 | 0.3 | 1.3 | 98.4 | 0.3 |
| week | | | | | | |
| 40 C. 2 | 1.5 | 98.1 | 0.4 | 1.6 | 98.0 | 0.4 |
| weeks | | | | | | |

There was therefore no difference between the behaviour of the two molecules under thermal stress.

### Example 3: TRAIL-R2-FcΔC Molecule Shows Improved Stability Under Low pH Stress

Material was thawed and both a concentration measurement and analytical size exclusion chromatography (aSEC) were carried out. The material was then buffer exchanged via dialysis into a low pH buffer, followed by normalisation and filtration using a 0.22 μm filter. Post filtration all the samples were thermally stressed at elevated temperature for 4 hours or two weeks. Samples were evaluated following incubation under stressed and unstressed conditions.

Molecules were subjected to analytical size exclusion chromatography (aSEC) to quantify the levels of (1) the percentage of high molecular weight species, (% HMwS) that is indicative of aggregation risk; (2) the percentage of low molecular weight species, (% HMwS) that is indicative of fragmentation risk; (3) the percentage monomer that is indicative of monomeric non-degraded species.

Table 4 shows a comparison between the Fc fusion proteins which had been subjected to low pH stress.

TABLE 4
TRAIL-R2-FcΔC molecule shows improved stability under low pH stress

| | | | SEC % (HMW/Monomer/ | |
|---|---|---|---|---|
| Molecule | Condition | Appearance | LMW(ΔMonomer)) | |
| TRAIL-R2-Fc | pH 3.5 - 4 hour | Appearance of | 6.4/93.6/ND | (−5.3) |
| | incubation | particulate matter | | |
| TRAIL-R2-Fc | pH 3.5 - 48 hour | Appearance of | 10.9/88.5/0.54 | (−10.4) |
| | incubation | particulate matter | | |
| TRAIL-R2-FcΔC | pH 3.5 - 4 hour | Normal | 7.5/95.6/ND | (−3.3) |
| | incubation | | | |
| TRAIL-R2-FcΔC | pH 3.5 - 48 hour | Normal | 7.6/92.1/ND | (−6.8) |
| | incubation | | | |

Thus improved stability under pH stress could be observed with the TRAIL-R2-FcΔC Fc fusion protein compared with the TRAIL-R2-Fc variant.

Further studies were conducted compare TRAIL-R2-FcΔC to TRAIL-R2-Fc C153S when placed under low pH stress. There were no significant differences between the behaviour of the two molecules under thermal stress.

### Example 4: TRAIL-R2-FcΔC molecule shows better stability compared to TRAIL-R2-Fc C153S under light oxidation stress

Material was thawed and both a concentration measurement and analytical size exclusion chromatography (aSEC) were carried out. The material was then exposed to light oxidation conditions for 1 day or 2 days. Samples were evaluated following incubation under stressed and unstressed conditions.

Table 5 shows a comparison between the TRAIL-R2-FcΔC and TRAIL-R2-Fc C153S molecules following light oxidation for either 1 or 2 days.

TABLE 5
TRAIL-R2-FcΔC molecule shows better stability compared
to TRAIL-R2-Fc C153S under light oxidation stress

| | TRAIL-R2-FcΔC | | | TRAIL-R2-Fc C153S | | |
|---|---|---|---|---|---|---|
| | % HMwS | % monomer | % LMwS | % HMwS | % monomer | % LMwS |
| Light - 1 D | 5.1 | 94.8 | 0.1 | 5.6 | 94.3 | 0.1 |
| Light - 2 D | 8.7 | 91.2 | 0.1 | 10.0 | 89.9 | 0.1 |

A 7.8% increase of HMW % was observed in TRAIL-R2-FcΔC protein, and 9.0% increase of HMW % was observed in TRAIL-R2-Fc C153S protein. Improved stability under light oxidation stress could be observed with the TRAIL-R2-FCΔC Fc fusion protein compared with the TRAIL-R2-Fc C153S variant.

### Example 5: Changing the Linker in a TRAIL-R2-Fc Fusion Protein Does Not Affect Biological Activity

In a 96-well plate, 10⁴ Hela cells were seeded per well in 100 ul of complete RPMI (supplemented with 10% FCS and 1% pen/strep). The following day, the seeding media was removed and the cells treated (in the same complete RPMI) with 100 ul of a combination of izhTRAIL and/or TRAIL-R2-Fc at the concentrations between 0-20 μg/ml, in triplicates (the izhTRAIL was pre-incubated with the TRAIL-R2-Fc for 1 h at room temperature before adding it on top of the cells). 24 h later, a viability assay was run and the viability of each condition normalized to that of the the 0 μg/ml izhTRAIL- and 0 μg/ml TRAIL-R2-Fc-treated control. The CellTiter-Glo assay from Promega was used. For the Cell Titer Glo: media was removed from the cells and 100 ul of the Cell Titer Glo solution added (diluted ⅕ in PBS). The mixture was incubated for 10 minutes in the dark and then 80 ul of the mix was transferred from each well to an opaque 96 well plate. The bioluminescence was read in a plate reader.

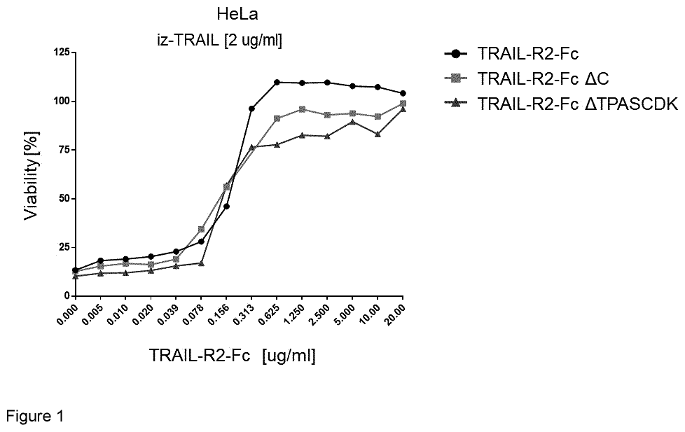

FIG. 1 shows the biological activity of three TRAIL-R2-Fc fusion proteins. FIG. 1 demonstrates that altering the linker does not affect the biological activity of the Fc fusion protein.

### Example 6: Conclusion

Optimisation of the TRAIL-R2-Fc sequence by removal of unpaired cysteines has led to improvements in: (1) expression of the protein; (2) thermal stability at 40C; (3) stability under low pH stress and (4) light stress conditions.

These improvements did not adversely affect the biological activity of the Fc fusion protein.

### Sequences Annex—Other Sequences Referred to Herein

a) Amino acid sequence of entire TRAIL-R2 and its splice variant

```
                10 20 30 40
MEQRGQNAPA ASGARKRHGP GPREARGARP GPRVPKTLVL
                50 60 70 80
VVAAVLLLVS AESALITQQD LAPQQRAAPQ QKRSSPSEGL
                90 100 110 120
CPPGHHISED GRDCISCKYG QDYSTHWNDL LFCLRCTRCD
              130 140 150 160
SGEVELSPCT TTRNTVCQCE EGTFREEDSP EMCRKCRTGC
              170 180 190 200
PRGMVKVGDC TPWSDIECVH KESGTKHSGE VPAVEETVTS
              210 220 230 240
SPGTPASPCS LSGIIIGVTV AAVVLIVAVF VCKSLLWKKV
              250 260 270 280
LPYLKGICSG GGGDPERVDR SSQRPGAEDN VLNEIVSILQ
              290 300 310 320
PTQVPEQEME VQEPAEPTGV NMLSPGESEH LLEPAEAERS
              330 340 350 360
QRRRLLVPAN EGDPTETLRQ CFDDFADLVP FDSWEPLMRK
              370 380 390 400
LGLMDNEIKV AKAEAAGHRD TLYTMLIKWV NKTGRDASVH
              410 420 430 440
TLLDALETLG ERLAKQKIED HLLSSGKFMY LEGNADSAMS
```

Short isoform (also known as TRICK2B) identifier O14763-2 (411 amino acids)

MEQRGQNAPAASGARKRHGPGPREARGARPGPRVPKTLVLVVAAVLLLVS
AESALITQQDLAPQQRAAPQQKRSSPSEGLCPPGHHISEDGRDCISCKYG
QDYSTHWNDLLFCLRCTRCDSGEVELSPCTTTRNTVCQCEEGTFREEDSP
EMCRKCRTGCPRGMVKVGDCTPWSDIECVHKESGIIIGVTVAAVVLIVAV
FVCKSLLWKKVLPYLKGICSGGGGDPERVDRSSQRPGAEDNVLNEIVSIL
QPTQVPEQEMEVQEPAEPTGVNMLSPGESEHLLEPAEAERSQRRRLLVPA
NEGDPTETLRQCFDDFADLVPFDSWEPLMRKLGLMDNEIKVAKAEAAGHR
DTLYTMLIKWVNKTGRDASVHTLLDALETLGERLAKQKIEDHLLSSGKFM
YLEGNADSAMS

b) Amino acid sequence of prior art TRAIL-R2-Fc protein sequence

The TRAIL-R2-Fc polypeptide from WO2015001345 is set out below.

The TRAIL-R2 portion is underlined. The Fc portion is depicted in bold. Note that there is a one amino acid overlap between the TRAIL-R2 portion and the human IgG1 Fc portion. The leader peptide is depicted in italics. The mature protein starts with the sequence ITQQDLA. When produced recombinantly, the exact position of the N terminus can vary by a few amino acids; that means the mature protein can be, e.g. one to five amino acids shorter or longer.

MEQRGQNAPAASGARKRHGPGPREARGARPGPRVPKTLVLVVAAVLLLVS
AESALITQQDLAPQQRAAPQQKRSSPSEGLCPPGHHISEDGRDCISCKYG
QDYSTHWNDLLFCLRCTRCDSGEVELSPCTTTRNTVCQCEEGTFREEDSP
EMCRKCRTGCPRGMVKVGDCTPWSDIECVHKESGTKHSGEVPAVEETVTS
SPGTPASCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPGK

c) Amino acid sequence and nucleotide sequence of TRAIL-R2-C153S-Fc (including leader) used in data comparisons

MGSTAILGLLLAVLQGVCAITQQDLAPQQRAAPQQKRSSPSEGLCPPGHH
ISEDGRDCISCKYGQDYSTHWNDLLFCLRCTRCDSGEVELSPCTTTRNTV
CQCEEGTFREEDSPEMCRKCRTGCPRGMVKVGDCTPWSDIECVHKESGTK
HSGEVPAVEETVTSSPGTPASSDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
K..

TRAIL-R2-C153S-Fc nucleotide sequence

ATGGGCTCCACCGCCATCCTGGGCCTGCTGCTGGCCGTGCTGCAGGGCGT
GTGCGCCATTACTCAGCAAGATCTGGCCCCTCAGCAAAGGGCCGCTCCAC
AACAGAAGAGAAGCAGCCCAAGCGAGGGACTCTGCCCTCCCGGCCACCAC
ATTAGCGAGGACGGCAGAGATTGCATCAGCTGTAAGTACGGCCAAGATTA
CAGCACACACTGGAATGATCTGCTGTTCTGTCTGAGGTGTACAAGGTGCG
ACTCCGGCGAAGTCGAACTGAGCCCTTGCACTACTACTAGGAACACTGTC
TGCCAGTGCGAGGAGGGCACTTTTAGGGAGGAGGATAGCCCAGAGATGTG
TAGGAAGTGTAGGACTGGCTGCCCTAGGGGCATGGTGAAGGTGGGCGACT
GTACTCCATGGAGCGACATTGAGTGCGTGCATAAGGAGAGCGGCACTAAG
CACAGCGGCGAAGTGCCAGCCGTGGAAGAGACTGTGACTAGCTCCCCCGG
CACACCAGCCAGCTCCGACAAGACACACACTTGTCCTCCTTGCCCAGCTC
CAGAACTGCTCGGAGGACCAAGCGTGTTTCTGTTCCCTCCAAAGCCAAAG
GACACACTCATGATCTCTAGGACTCCAGAGGTCACTTGCGTGGTCGTGGA
TGTCAGCCACGAAGATCCAGAGGTGAAGTTTAATTGGTACGTGGATGGCG
TGGAGGTCCACAACGCCAAGACAAAGCCTAGGGAGGAGCAGTACAATAGC
ACTTATAGGGTGGTCAGCGTGCTGACAGTGCTGCACCAAGATTGGCTCAA
CGGCAAGGAGTACAAGTGCAAGGTGAGCAACAAGGCCCTCCCAGCCCCTA
TCGAGAAGACAATCTCCAAGGCCAAGGGCCAACCAAGGGAGCCTCAAGTG
TATACACTCCCTCCTAGCAGAGACGAGCTCACTAAGAATCAAGTGTCTCT
GACTTGTCTGGTGAAGGGCTTTTACCCAAGCGACATCGCCGTGGAGTGGG
AGAGCAACGGCCAACCAGAGAACAACTACAAGACAACTCCTCCAGTGCTG
GACTCCGACGGCAGCTTCTTCCTCTACAGCAAGCTGACTGTGGATAAGTC
CAGATGGCAGCAAGGCAACGTCTTCAGCTGTAGCGTGATGCACGAGGCCC
TCCACAACCACTACACACAGAAGTCTCTGTCTCTGAGCCCCGGCAAATAA
TAG

d) Amino acid sequence and nucleotide sequence of TRAIL-R2-Fc ΔTPASCDK used in data comparisons

ITQQDLAPQQRAAPQQKRSSPSEGLCPPGHHISEDGRDCISCKYGQDYST
HWNDLLFCLRCTRCDSGEVELSPCTTTRNTVCQCEEGTFREEDSPEMCRK
CRTGCPRGMVKVGDCTPWSDIECVHKESGTKHSGEVPAVEETVTSSPGTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFS
CSVMHEALHNHYTQKSLSLSPGK..

Nucleotide sequence

ATTACTCAGCAAGATCTGGCCCCTCAGCAAAGGGCCGCTCCACAACAGAA
GAGAAGCAGCCCAAGCGAGGGACTCTGCCCTCCCGGCCACCACATTAGCG
AGGACGGCAGAGATTGCATCAGCTGTAAGTACGGCCAAGATTACAGCACA
CACTGGAATGATCTGCTGTTCTGTCTGAGGTGTACAAGGTGCGACTCCGG
CGAAGTCGAACTGAGCCCTTGCACTACTACTAGGAACACTGTCTGCCAGT
GCGAGGAGGGCACTTTTAGGGAGGAGGATAGCCCAGAGATGTGTAGGAAG
TGTAGGACTGGCTGCCCTAGGGGCATGGTGAAGGTGGGCGACTGTACTCC
ATGGAGCGACATTGAGTGCGTGCATAAGGAGAGCGGCACTAAGCACAGCG
GCGAAGTGCCAGCCGTGGAAGAGACTGTGACTAGCTCCCCCGGCACACAC
ACTTGTCCTCCTTGCCCAGCTCCAGAACTGCTCGGAGGACCAAGCGTGTT
TCTGTTCCCTCCAAAGCCAAAGGACACACTCATGATCTCTAGGACTCCAG
AGGTCACTTGCGTGGTCGTGGATGTCAGCCACGAAGATCCAGAGGTGAAG
TTTAATTGGTACGTGGATGGCGTGGAGGTCCACAACGCCAAGACAAAGCC
TAGGGAGGAGCAGTACAATAGCACTTATAGGGTGGTCAGCGTGCTGACAG
TGCTGCACCAAGATTGGCTCAACGGCAAGGAGTACAAGTGCAAGGTGAGC
AACAAGGCCCTCCCAGCCCCTATCGAGAAGACAATCTCCAAGGCCAAGGG
CCAACCAAGGGAGCCTCAAGTGTATACACTCCCTCCTAGCAGAGACGAGC
TCACTAAGAATCAAGTGTCTCTGACTTGTCTGGTGAAGGGCTTTTACCCA
AGCGACATCGCCGTGGAGTGGGAGAGCAACGGCCAACCAGAGAACAACTA
CAAGACAACTCCTCCAGTGCTGGACTCCGACGGCAGCTTCTTCCTCTACA
GCAAGCTGACTGTGGATAAGTCCAGATGGCAGCAAGGCAACGTCTTCAGC
TGTAGCGTGATGCACGAGGCCCTCCACAACCACTACACACAGAAGTCTCT
GTCTCTGAGCCCCGGCAAATAATAG..

### References

Bozkurt, E., Dussman, H., Salvucci, M., Cavanagh, B. L., van Schaeybroeck, S., Longley, D. B., Martin, S. J and Prehn, J. H. M. (2021) TRAIL signaling promotes entosis in colorectal cancer. J Cell Biol 220 (11), ePub.

Ethiraj P., Sambandam, Y., Hathaway-Schrader, J. D., Haque, A., Novince, C. M and Reddy, S. V. (2020) RANKL triggers resistance to TRAIL-induced death in oral squamous cell carcinoma. J Cell Physiol 235 (20), 1663-1673.

Yan, S., Qu, X., Xu, C., Zhu, Z., Zhang, L., Xu, L., et al., (2012) Down-regulation of Cbl-b by bufalin results in up-regulation of DR4/DR5 and sensitization of TRAIL-induced apoptosis in breast cancer cells. J Cancer Res Clin Oncol. 138 (8), 1279-1289.

Hameed, A. G., Arnold, N. D., Chamberlain, J, Pickworth, J. A., Paiva, C., Dawson, S. et al. (2012) Inhibition of tumor-necrosis factor-related apoptosis-inducing ligand (TRAIL) reverses experimental pulmonary arterial hypertension. JEM 209 (11) 1919-1935.

Lawrie, A. (2014) The role of the osteoprotegerin/tumor necrosis factor related apoptosis-inducing ligand axis in the pathogenesis of pulmonary arterial hypertension. Vascul. Pharmacol. 63 (3) 114-117.

Hirsova, P., Weng, P., Salim, W., Bronk, S. F., Griffith, T. S., Ibrahim, S. H., Gores, G. J. (2017) TRAIL deletion prevents liver inflammation but not adipose tissue inflammation during murine diet-induced obesity. Hept Comms. 1 (7) 648-662.

Farrell, G. C., Larter, C. Z., Hou, J. Y., Zhang, R. H., Yeh, M. M., Williams, J., dela Pena, A., et al. (2008) Apoptosis in experimental NASH is associated with p53 activation and TRAIL receptor expression. J Gastroenterol Hepatol. 24 (3) 443-452.

Valiente, A. J., liem, K. S., Schwartz, K. B., Rosenthal, P., Murray, K. F., Mogul, D., Teckman, J., et al., (2021) The Inflammatory Cytokine Profile Associated with Liver Damage is Broader and Stronger in Chronic Hepatitis B Patients Compared to Acute Hepatitis B Patients J Infect Dis. Epub.

Qin L, Zou J, Barnett A. Vetreno R. P. Crews F. T, Coleman L. G. (2021) TRAIL mediates neuronal death in AUD: a link between neuroinflammation and neurodegeneration. Int J Mol Sci. 22 (5) 2547

Jeong, D., Kim, H. S., Kim, H. Y., Kang, M. J., Jung, H., Oh, y., kim, D., Kohn, J., et al., (2021) Soluble Fas ligand drives autoantibody-induced arthritis by binding to DR5/TRAIL-R2. ELife 10.

Wu, Y., kimura, Y., Okamoto, T., Matsuhisa, K., Asada, r., Saito, A., Sakaue, F., Kazunori, K and Kaneko, M. (2019) inflammatory bowel disease-associated ubiquitin ligase RNF183 promots lysosomal degradation of DR5 and TRAIL-induced caspase activation. Sci Rep. 9 (1) 20301.

Starkey, M. R., Nguyen, D. H., Essilifie, A. T., Kim, R. Y., Hatchwell, L. M, Collison, A. M., Yagita, H., et al., (2014) Tumor tumor necrosis factor related apoptosis-inducing ligand translates neonatal respiratory infection into chronic lung disease. Mucosal Immunology. 7, 478-488.

Ashkenazi, A., Pai, R. C., Fong, S., Leung, S., Lawrence, D. A., Marsters, S. A., Blackie, C., Chang, L., McMurtrey, A. E., Hebert, A., et al. (1999). Safety and antitumor activity of recombinant soluble Apo2 ligand. J Clin Invest 104, 155-162.

Walczak, H., Miller, R. E., Ariail, K., Gliniak, B., Griffith, T. S., Kubin, M., Chin, W., Jones, J., Woodward, A., Le, T., et al. (1999). Tumoricidal activity of tumor necrosis factor-related apoptosis-inducing ligand in vivo. Nat Med 5, 157-163.

Screaton, G. R., J. Mongkolsapaya, X. N. Xu, A. E. Cowper, A. J. McMichael, and J. I. Bell. 1997. ‘TRICK2, a new alternatively spliced receptor that transduces the cytotoxic signal from TRAIL’, Current biology: CB, 7:693-96.

Hidalgo, M. (2010). Pancreatic cancer. The New England journal of medicine 362, 1605-1617.

Jaffee, E. M., Hruban, R. H., Canto, M., and Kern, S. E. (2002). Focus on pancreas cancer. Cancer cell 2, 25-28.

Grady, W. M., and Markowitz, S. D. (2002). Genetic and epigenetic alterations in colon cancer. Annu Rev Genomics Hum Genet 3, 101-128.

Mitsuuchi, Y., and Testa, J. R. (2002). Cytogenetics and molecular genetics of lung cancer. Am J Med Genet 115, 183-188.

Ito, K., Watanabe, K., Nasim, S., Sasano, H., Sato, S., Yajima, A., Silverberg, S. G., and Garrett, C. T. (1996). K-ras point mutations in endometrial carcinoma: effect on outcome is dependent on age of patient. Gynecologic oncology 63, 238-246.

Wegman, P., Ahlin, C., and Sorbe, B. (2011). Genetic alterations in the K-Ras gene influence the prognosis in patients with cervical cancer treated by radiotherapy. International journal of gynecological cancer: official journal of the International Gynecological Cancer Society 21, 86-91.

Przybojewska, B., Jagiello, A., and Jalmuzna, P. (2000). H-RAS, K-RAS, and N-RAS gene activation in human bladder cancers. Cancer genetics and cytogenetics 121, 73-77.

Boix-Ferrero, J., Pellin, A., Blesa, J. R., Adrados, M., and Llombart-Bosch, A. (2000). K-ras Gene Mutations in Liver Carcinomas from a Mediterranean Area of Spain. International journal of surgical pathology 8, 267-270.

Ahmad, E. I., Gawish, H. H., Al-Azizi, N. M., and El-Hefni, A. M. (2009). The Prognostic Impact of K-RAS Mutations in Adult Acute Myeloid Leukemia Patients Treated with High Dose Cytarabine. Journal of the Egyptian National Cancer Institute 21, 343-350.

Karnoub, A. E., and Weinberg, R. A. (2008). Ras oncogenes: split personalities. Nature reviews Molecular cell biology 9, 517-531.

Amado, R. G., Wolf, M., Peeters, M., Van Cutsem, E., Siena, S., Freeman, D. J., Juan, T., Sikorski, R., Suggs, S., Radinsky, R., et al. (2008). Wild-type KRAS is required for panitumumab efficacy in patients with metastatic colorectal cancer. Journal of clinical oncology: official journal of the American Society of Clinical Oncology 26, 1626-1634.

Deschoolmeester, V., Boeckx, C., Baay, M., Weyler, J., Wuyts, W., Van Marck, E., Peeters, M., Lardon, F., and Vermorken, J. B. (2010). KRAS mutation detection and prognostic potential in sporadic colorectal cancer using high-resolution melting analysis. British journal of cancer 103, 1627-1636.

Karapetis, C. S., Khambata-Ford, S., Jonker, D. J., O'Callaghan, C. J., Tu, D., Tebbutt, N. C., Simes, R. J., Chalchal, H., Shapiro, J. D., Robitaille, S., et al. (2008). K-ras mutations and benefit from cetuximab in advanced colorectal cancer. The New England journal of medicine 359, 1757-1765.

van Krieken, J. H., Jung, A., Kirchner, T., Carneiro, F., Seruca, R., Bosman, F. T., Quirke, P., Flejou, J. F., Plato Hansen, T., de Hertogh, G., et al. (2008). KRAS mutation testing for predicting response to anti-EGFR therapy for colorectal carcinoma: proposal for an European quality assurance program. Virchows Archiv: an international journal of pathology 453, 417-431.

Downward, J. (2003). Targeting RAS signalling pathways in cancer therapy. Nat Rev Cancer 3, 11-22.

Chaffer, C. L., and Weinberg, R. A. (2011). A perspective on cancer cell metastasis. Science 331, 1559-1564.

Ahn, J., Sanz-Moreno, V., and Marshall, C. J. (2010). The metastasis gene NEDD9 product acts through integrin beta3 and Src to promote mesenchymal motility and inhibit amoeboid motility. J Cell Sci 125, 1814-1826.

Klein, R. M., Spofford, L. S., Abel, E. V., Ortiz, A., and Aplin, A. E. (2008). B-RAF regulation of Rn3 participates in actin cytoskeletal and focal adhesion organization. Mol Biol Cell 19, 498-508.

Ehrenreiter, K., Kern, F., Velamoor, V., Meissl, K., Galabova-Kovacs, G., Sibilia, M., and Baccarini, M. (2009). Raf-1 addiction in Ras-induced skin carcinogenesis. Cancer cell 16, 149-160.

Belin de Chantemele, E. J., Retailleau, K., Pinaud, F., Vessieres, E., Bocquet, A., Guihot, A. L., Lemaire, B., Domenga, V., Baufreton, C., Loufrani, L., et al. (2008). Notch3 is a major regulator of vascular tone in cerebral and tail resistance arteries. Arteriosclerosis, thrombosis, and vascular biology 28, 2216-2224.

Gonzalez de Castro, D., Angulo, B., Gomez, B., Mair, D., Martinez, R., Suarez-Gauthier, A., Shieh, F., Velez, M., Brophy, V. H., Lawrence, H. J., et al. (2012). A comparison of three methods for detecting KRAS mutations in formalin-fixed colorectal cancer specimens. British journal of cancer 107, 345-351.

1. A fusion protein comprising, consisting essentially of, or consisting of the amino acid sequence of Seq. ID No: 1.

2. The fusion protein of claim 1, wherein the fusion protein lacks an N-terminal signal sequence.

3. The fusion protein of claim 1, wherein the fusion protein comprises an N-terminal signal sequence.

4. The fusion protein of claim 3, comprising an amino acid sequence as shown in Seq. ID No: 2 or Seq. ID No: 3.

5. A method of therapeutic treatment of a disease in an individual, the method comprising administering to the individual a therapeutically-effective amount of the fusion protein of claim 1.

6. The method of claim 5, wherein the individual is a human subject.

7. The method of claim 5, wherein the disease is cancer.

8. The method of claim 7, wherein the cancer is a KRAS-mutated cancer.

9. The method of claim 7, wherein the cancer is a cancer in which ROCK is inhibited independently of KRAS mutation.

10. The method of claim 9, wherein the cancer is one which is associated with aberrant signalling in a pathway selected from the group consisting of Src, FAK, BRAF, Raf-1, and Notch3.

11. The method of claim 8, wherein said cancer is selected from the list consisting of: pancreatic cancer, colorectal cancer, lung cancer, breast cancer, endometrial cancer, cervical cancer, liver cancer, myeloid leukemia, cholangiocarcinoma, and bladder cancer.

12. The method of claim 7, wherein said cancer is selected from colorectal cancer, squamous cell carcinoma, breast cancer and non-small cell lung cancer.

13. The method of claim 7, wherein said method inhibits or prevents activation of Rac1 by TRAIL and/or a TRAIL-Receptor in the cancer.

14. The method of claim 7, wherein said fusion protein is administered in combination with a further anti-cancer agent.

15. The method of claim 5, wherein in the disease is selected from the list consisting of: pulmonary arterial hypertension; non-alcoholic steatohepatitis and other liver disease; neurodegeneration and neuroinflammation; inflammatory disease, which is optionally inflammatory bowel disease, Crohn's disease or ulcerative colitis; and chronic lung disease.

16.-18. (canceled)

19. A pharmaceutical composition comprising the fusion protein of claim 1 and a pharmaceutically acceptable carrier, diluent, or excipient.

20. A nucleic acid molecule encoding the fusion protein of claim 1.

21. The nucleic acid molecule of claim 20, wherein the nucleic acid molecule is operably linked to an expression control sequence.

22. The nucleic acid molecule of claim 21, wherein the nucleic acid molecule is located on a vector.

23. A cell transformed or transfected with the nucleic acid molecule claim 20.

24. The cell of claim 23, wherein the cell is a prokaryotic cell.

25. The cell of claim 23, wherein the cell is a eukaryotic cell, which is optionally a mammalian cell.

26.-28. (canceled)
